(19) United States
(12) Patent Application Publication
(10) Pub. No.: US 2026/0334762 A1
(43) Pub. Date: Oct. 29, 2026

(54) ENDCAP FOR OVER THE SCOPE CLIP

(71) Applicant: BOSTON SCIENTIFIC SCIMED, INC., Maple Grove, MN (US)

(72) Inventors: Vladimir MITELBERG, Austin, TX (US); Ryan Davis GILKEY, Austin, TX (US); Alexander Ross COHEN, Austin, TX (US); Caroline Cooper GAUDET, Malden, MA (US)

(21) Appl. No.: 19/631,328

(22) Filed: Mar. 27, 2026

Related U.S. Application Data
(60) Provisional application No. 63/796,098, filed on Apr. 28, 2025.

(51) Int. Cl. A61B 1/00; A61B 17/08; A61B 17/10

(52) U.S. Cl. CPC A61B 1/0014; A61B 1/00137; A61B 17/083; A61B 17/10

(57) ABSTRACT

An endcap device for clipping tissue includes a body extending longitudinally from a proximal end to a distal end and including a channel extending longitudinally therethrough, a proximal portion of the body sized shaped and configured to receive a distal end of an endoscope therein, and distal portion of the body sized and shaped so that a clip is mountable thereover, in an open configuration; and a pair of clamp pads movably received within openings extending laterally through a wall along the proximal portion of the body, the pair of clamp pads movable from a first position in which the endoscope is receivable within the channel of the body between the pair of clamp pads, and a second position, in which the clamp pads are moved radially inward so that a portion thereof extend into the channel to grip a portion of the endoscope receivable therebetween.

### PRIORITY CLAIM

The present disclosure claims priority to U.S. Provisional Patent Application Ser. No. 63/796,098 filed Apr. 28, 2025; the disclosure of which is incorporated herewith by reference.

### FIELD

The present disclosure relates to endoscopic devices and, in particular, relates to endoscopic clipping devices for treating tissue along the gastrointestinal tract.

### BACKGROUND

Physicians have become more willing to perform aggressive interventional and therapeutic endoscopic gastrointestinal (GI) procedures, which may increase the risk of perforating the wall of the GI tract or may require closure of the GI tract wall as part of the procedure. Such procedures may include, for example, the removal of large lesions, tunneling under the mucosal layer of the GI tract to treat issues below the mucosa, full thickness removal of tissue, treatment of issues on other organs by passing outside of the GI tract, and endoscopic treatment/repair of post-surgical issues (e.g., post-surgical leaks, breakdown of surgical staple lines, and anastomotic leaks). Currently, tissue may be treated via endoscopic closure devices including through-the scope clips or over-the-scope clips.

Over-the-scope clips (OTSC) may be particularly useful for achieving closure of larger tissue defects. These clips are generally navigated to a target region in a human body in an open configuration, in which the jaws of the clip extend over an end cap that is mounted over a distal end of a flexible endoscope so that the jaws are separated from one another. The clip may then be moved toward a closed configuration by moving the clip distally off of the end cap so that the clip jaws move toward one another, around a target tissue, under a natural bias thereof. In some cases, a placement of the clip may be reviewed before final deployment of the clip. If an operator (e.g., physician) determines that the clip is not placed over target tissue, as desired, the clip may be drawn proximally over the end cap so that the jaws are once again moved to an open configuration and the clip may be repositioned over the target tissue. These repositionable clips may be repeatedly moved between the open and closed configurations, as desired, until it is determined that the clip has been clipped over the target tissue, in a desired configuration. In some cases, however, repeated movement of the clip between the open and closed configurations may affect the retention of the endcap on the distal end of the endoscope, making it difficult to reposition the clip.

### SUMMARY

The present disclosure relates to an endcap device for clipping tissue. The device includes a body extending longitudinally from a proximal end to a distal end and including a channel extending longitudinally therethrough, a proximal portion of the body sized shaped and configured to receive a distal end of an endoscope therein, and distal portion of the body sized and shaped so that a clip is mountable thereover, in an open configuration; and a pair of clamp pads movably received within openings extending laterally through a wall along the proximal portion of the body, the pair of clamp pads movable from a first position in which the endoscope is receivable within the channel of the body between the pair of clamp pads, and a second position, in which the clamp pads are moved radially inward so that a portion thereof extend into the channel to grip a portion of the endoscope receivable therebetween.

In an embodiment, the device further includes a joint member sized, shaped, and configured to be positioned along a portion of an exterior surface of the endoscope, proximally of the body; and a pair of tubular structures connecting the joint member to the proximal portion of the body, the pair of tubular structures configured to receive control members for controlling a movement of a clip mounted over the distal portion of the body.

In an embodiment, the device further includes a seal extending in the channel of the body, distally of the pair of clamp pads, the seal configured to seal the channel of the body against a distal face of the endoscope so that a vacuum force may be applied therethrough.

In an embodiment, the seal includes one of a seal gasket and a lip valve.

In an embodiment, the proximal portion includes a tape hook along an exterior surface thereof, between the pair of clamp pads and in alignment therewith.

In an embodiment, the joint member includes a tape hook configured to engage an end of a tape to facilitate attachment of the joint member to the exterior surface of the endoscope.

In an embodiment, each of the pair of clamp pads extends along a curve corresponding to a curvature along a portion of the channel of the body.

In an embodiment, an interior surface of each of the pair of clamps includes gripping structures for engaging a portion of the endoscope received therebetween.

In an embodiment, the distal portion of the body tapers from a proximal end thereof to a distal end thereof and includes grooves extending longitudinally along an exterior surface thereof.

In addition, the present disclosure relates to a clipping system for treating tissue. The system includes an endoscope extending longitudinally from a proximal end to a distal end; an endcap including a body extending longitudinally from a proximal end to a distal end, the body including a channel extending longitudinally therethrough, a proximal portion of the body sized shaped and configured to receive the distal end of the endoscope therein and a distal portion of the body an insertion device therein, a pair of clamp pads movably received within openings extending laterally through a wall of the proximal portion of the body, the pair of clamp pads movable from a first position in which the endoscope is receivable within the channel of the body between the pair of clamp pads, and a second position, in which the clamp pads are moved radially inward to grip a portion of the endoscope received therebetween; and a clip mountable over a distal portion of the body of the endcap, in an open configuration, in which jaws of the clip extend over opposing sides of the distal portion of the endcap so that the jaws are separated from one another, the jaws of the clip movable between the open configuration, and a closed configuration, in which the jaws are moved toward one another, the pair of jaws connected to one another via hinges biasing the clip toward the closed configuration.

In an embodiment, the endcap further includes a joint member connected to the proximal portion of the body via a pair of tubular structures, the tubular structures extending from proximal ends, connected to one another via the joint member to form a substantially Y-shape, to distal ends connected to the proximal portion of the body.

In an embodiment, the system further includes a pair of control members extending from proximal ends accessible to an operator of the system, along a length of the endoscope, through the pair of tubular structures to a distal end releasably connected to jaws of the clip to move the clip between the open configuration, the closed configuration, and a final deployed configuration.

In an embodiment, the joint member includes a tape hook configured to engage an end of a tape to facilitate attachment of the joint member to an exterior surface of the endoscope.

In an embodiment, the endcap includes a seal extending in the channel of the body, distally of the pair of clamp pads, to seal the channel of the body against a distal face of the endoscope so that a vacuum force may be applied therethrough.

In an embodiment, the proximal portion of the body of the endcap includes a tape hook along an exterior surface thereof, between the pair of clamp pads, in alignment therewith.

In addition, the present disclosure relates to a method for treating tissue. The method includes mounting an endcap over a distal end of an endoscope so that the distal end of the endoscope is received within a channel of a body of the endcap via a proximal portion of the endcap; moving clamp pads received within openings through a wall of the proximal portion of the body of the endcap from a first position, in which the distal end of the endoscope is receivable within the channel between the clamp pads, and a second position, in which the clamp pads extend into the channel to grip the endoscope; and fixing the clamp pads toward the second position so that the endcap is fixed over the distal end of the endoscope.

In an embodiment, moving the clamp pads from the first position toward the second position and fixing the clamp pads toward the second position includes winding a length of tape about the proximal portion of the body, about the clamp pads, to move and maintain the clamp pads in a radially inward configuration.

In an embodiment, the tape is wound about the proximal portion by hooking an end of the tape to a tape hook along an exterior of the proximal portion so that the tape is tensioned as the tape is wound about the proximal portion of the body.

In an embodiment, the method further includes fixing a joint member of the endcap directly to the endoscope, proximally of the body, the joint member connected to the body of the endcap via tubular structures configured to receive control members for controlling a movement of a clip relative to the endcap.

In an embodiment, the method further includes inserting the endcap, with a clip mounted over a distal portion thereof in an open configuration, to a target area within a body lumen; drawing target tissue between jaws of the clip via a suction force applied through the endoscope and through the channel of the body of the endcap; moving the clip between the open configuration, and a closed configuration, in which the clip is moved distally off of the distal portion of the body of the endcap so that the jaws move toward one another under a natural bias, wherein movement of the clip between the open and closed configurations is controlled via control members extending from proximal ends, along a length of the endoscope, through the tubular structure of the endcap to distal ends releasably coupled to the clip; and finally deploying the clip, when it is determined that the clip is clipped over the target tissue as desired, by releasing the clip from the control members.

### BRIEF DESCRIPTION

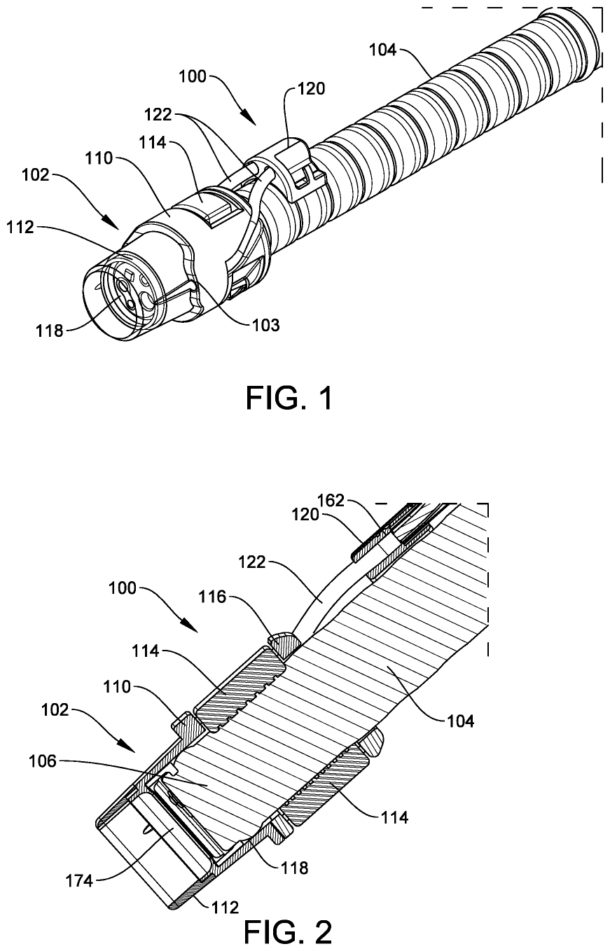

FIG. 1 shows a perspective view of a distal portion of a system according to an exemplary embodiment of the present disclosure;

FIG. 2 shows a longitudinal cross-section view of the distal portion of the system according to FIG. 1;

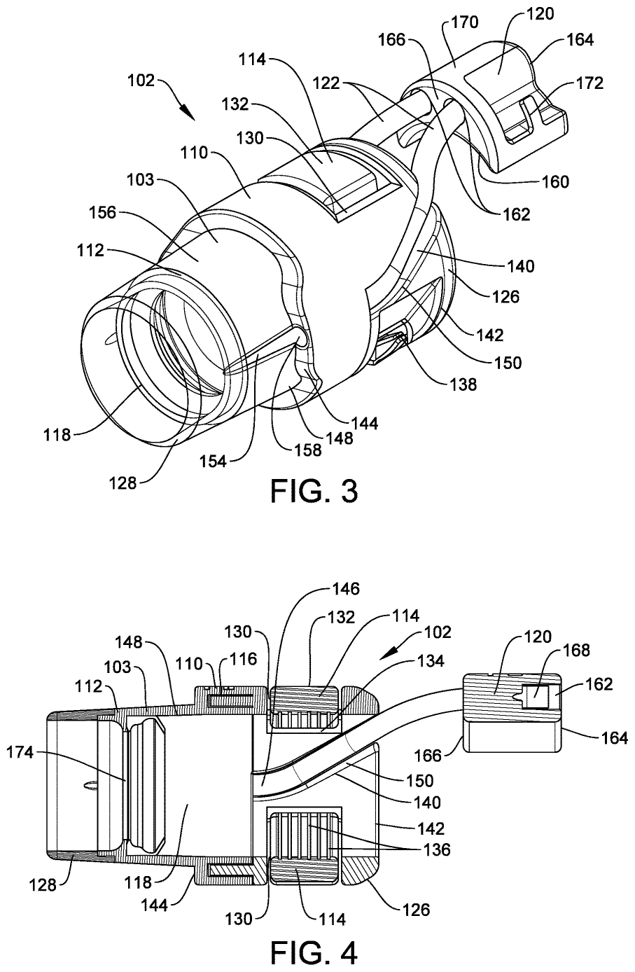

FIG. 3 shows a perspective view of an endcap of the system according to the exemplary system of FIG. 1;

FIG. 4 shows a partial cross-sectional view of the endcap according to the exemplary system of FIG. 1;

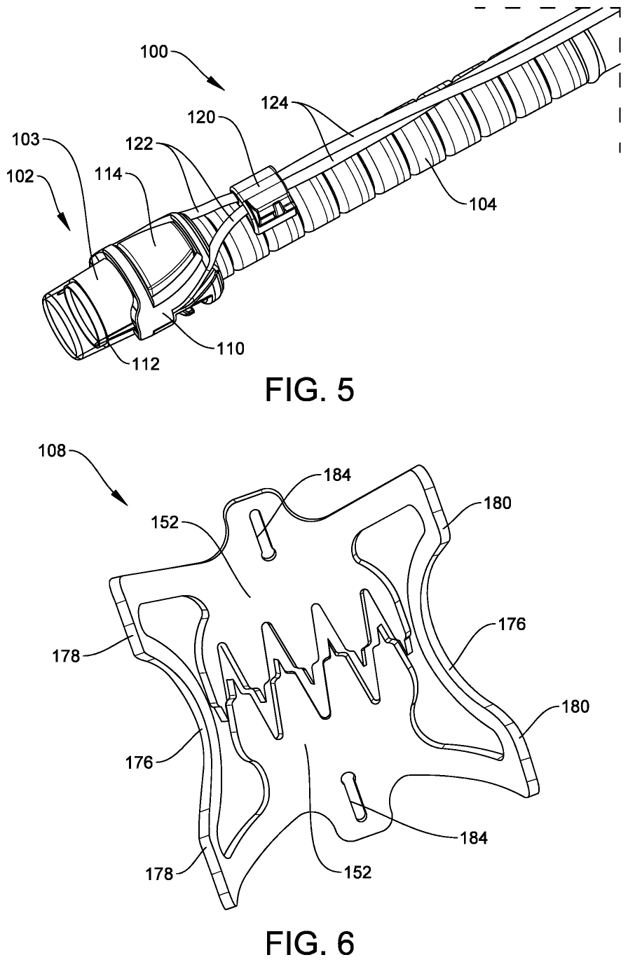

FIG. 5 shows another perspective view of the distal portion of the system according to FIG. 1;

FIG. 6 shows a perspective view of a clip according to the exemplary system of FIG. 1;

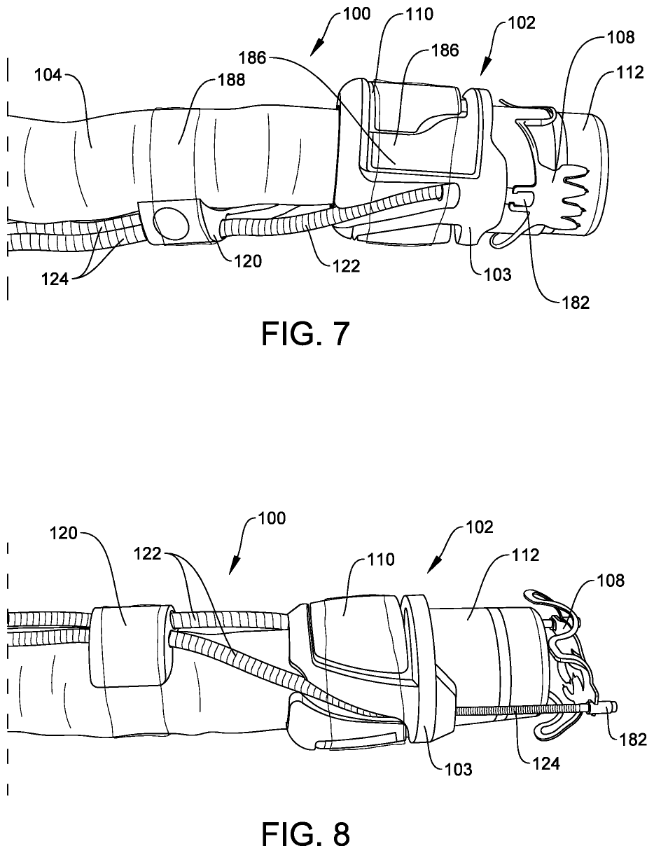

FIG. 7 shows a longitudinal side view of the distal portion of the system according to FIG. 1, with the clip mounted over the endcap in an open configuration;

FIG. 8 shows a longitudinal side view of a distal portion of the system according to FIG. 1, with the clip in an initial closed configuration.

### DETAILED DESCRIPTION

The present disclosure may be further understood with reference to the following description and the appended drawings, wherein like elements are referred to with the same reference numerals. The present disclosure relates to a clipping system and, in particular, relates to an endoscopic clipping system. Exemplary embodiments of the present disclosure describe a system comprising an endcap configured to be mounted over a distal end of an endoscope to facilitate application of a device such as an over-the-scope clip.

The endcap includes a pair of clamp pads movably received within a wall of the endcap, the pair of clamp pads configured to be moved radially inward so that a portion of the clamp pads extends into a channel of the endcap to grip a portion of the endoscope received therein. Tape may be wound about the endcap, over the clamp pads, to maintain the clamp pads in this gripping configuration so that the endcap is fixed over the endoscope. The endcap may additionally include a seal housed within a channel thereof where the seal is configured to engage a distal face of the endoscope received therein so that vacuum force applied through the endcap is maintained within the endcap (i.e., does not leak out of a gap or gaps between the endcap and the scope) to facilitate a suction of target tissue into the endcap.

It will be understood by those of skill in the art that although the exemplary embodiments show and describe the endcap mounted over the distal end of an endoscope, the exemplary endcap may be similarly utilized with other scope devices and/or insertion devices having an elongate shaft that may be used to deliver a clip. It will also be understood by those of skill in the art that although the exemplary endcap is specifically described with respect to the application of over the scope clips, the exemplary endcap may be similarly utilized for other endoscopic procedures in which tissue acquisition is required. It will be understood by those of skill in the art that the terms “proximal” and “distal,” as used herein, are intended to refer to a direction toward and away from, respectively, a user of the device. Furthermore, it will be understood that, although the endcaps of the present embodiments are described in relation to systems using over the scope clips, that these endcaps can be used with any device for which an endcap is to be placed over a round device such as any type of scope or other tubular access device for the which it is desired to enhance a seal between the endcap and the round device.

As shown in FIGS. 1-8, a clipping system 100 according to an exemplary embodiment of the present disclosure comprises an endcap 102 configured to be mounted over a distal end 106 of an endoscope 104 (or other access device) to facilitate application of a clip 108 (as shown in FIG. 6) over target tissue. The endcap 102 includes a body 103 having a proximal portion 110 configured to be mounted over the endoscope 104, as shown in FIGS. 1-2, and a distal portion 112 configured to project distally beyond a distal end of the endoscope 104. As would be understood, the distal portion 112 may accommodate the clip 108 mounted thereover, in an open configuration. Alternatively, the clip 108 may be mounted over the proximal portion 110 so that the clip 108 is advanced over and off of the distal portion 112 when deployed.

According to an exemplary embodiment, the proximal portion 110 includes a pair of clamp pads 114 movably received within a portion of a wall 116 of the body 103. The clamp pads 114 may be movable between a first position, in which the clamp pads 114 are positioned (e.g., withdrawn radially outward) to facilitate insertion of the distal end 106 of the endoscope 104 into a channel 118 of the body 103 of the endcap 102 until the proximal portion 110 is seated on the endoscope 104 as desired. The clamp pads 114 are movable to a second position in which the clamp pads 114 are moved radially inward to extend into the channel 118 to grip a portion of the endoscope 104 received therein.

In an exemplary embodiment, tape is wound over the clamp pads 114 to push the clamp pads 114 radially inward from the first position to the second position. The tape may then remain in place around the endcap 102 maintain the clamp pads 114 in the second position fixing the endcap 102 in a desired position on the distal end 106 of the endoscope 104. The endcap 102 may additionally include a seal 174 within the channel 118 to seal the body 103 of the endcap 102 against the distal end 106 of the endoscope 104 so that vacuum force applied into the channel 118 to draw a target tissue therein is maintained within the endcap and does not leak out of the proximal end 126 thereof.

The seal 174 and the clamp pads 114 permit the endcap 102 to accommodate endoscopes 104 of varying geometries while still providing a seal which allows for the effective application of vacuum force therethrough. In exemplary embodiment, the endcap 102 may additionally include a joint member 120 connected to the body 103 via a pair of tubular structures 122 configured to receive control members 124 therethrough where the control members 124 are coupled to the clip 108 so that manipulation of the control members 124 controls movement of the clip 108 relative to the endcap 102, as will be described in further detail below.

As shown in FIGS. 3-4, the body 103 of the endcap 102 extends longitudinally from a proximal end 126 to a distal end 128 and includes the channel 118 extending longitudinally therethrough. As described above, the proximal portion 110 of the body 103 is configured to be mounted over the distal end 106 of the endoscope 104 while the distal portion 112 of this embodiment is configured to accommodate the clip 108 stretched thereover in the open configuration. The proximal portion 110 includes a pair of openings 130 which in this embodiment extend laterally through the wall 116 of the distal portion 110 so that the channel 118 is opened to an exterior of the body 103 via the openings 130 but with the clamp pads 114 sealing the openings 130. In an exemplary embodiment, the openings 130 are positioned at diametrically opposed portions of the body 103. Each of the openings 130 is sized, shaped and configured to receive a corresponding one of the clamp pads 114 therein so that the clamp pad 114 is movable therein, between the first position and the second position while sealing the openings 130 so that the vacuum force is maintained within the channel 118.

In the first position, the clamp pads 114 are moved radially outward so the clamp pads 114 do not interfere with the insertion of the distal end 106 of the endoscope 104 as it is slid into the channel 118. When the endoscope 104 is positioned within the endcap 102 as desired, the clamp pads 114 are moved from the first position to press against the endoscope 104 in the second position. The user moves the clamp pads 114 to the second position by applying a radially compressive force to the clamp pads 114 by, for example, wrapping a flexible member such as a tape around the proximal portion 110 of the endcap 102. In the second position, a portion of each of the clamp pads 114 extends into the channel 118 to grip a portion of the endoscope 104 received therebetween. In an exemplary embodiment, the clamp pads 114 are diametrically opposed to one another so that, when moved toward the second position, the clamp pads 114 engage opposing sides of the endoscope 104, fixing the endoscope 104 concentrically in the channel 118 of the endcap 102.

As described above, each of the clamp pads 114 is received within a corresponding one of the openings 130. Each of the clamp pads 114 extends about a curve substantially corresponding to a curvature of a portion of the wall 116 of the body 103 through which the clamp pad 114 extends so that, in an exemplary embodiment, the wall 116 and the clamp pads 114 form a substantially continuous curved (e.g., cylindrical) shape. Each of the clamp pads 114 defines an exterior surface 132 and an interior surface 134. The exterior surface 132 is accessible from an exterior of the body 103 of the endcap 102 while the interior surface 134 is accessible from an interior of the channel 118 to grip the endoscope 104 received therein as described above. In an exemplary embodiment, the interior surface 134 includes ridges 136, teeth, or other gripping structures to facilitate engagement with the endoscope 104, when the endoscope 104 is gripped between the clamp pads 114.

The clamp pads 114 may be movably coupled to the body 103 via any of a variety of mechanisms. In one exemplary embodiment, each of the clamp pads 114 is coupled to the body 103 via one or more pins extending longitudinally through the wall 116 and through a longitudinal opening extending through the clamp pad 114. It will be understood by those of skill in the art that the longitudinal channel should be elongated along a lateral axis (e.g., an axis extending substantially perpendicular to a longitudinal axis of the body 103) to allow the clamp pad 114 to move radially (toward and away from a longitudinal axis of the body 103) relative to the body 103 of the endcap 102. It will be understood by those of skill in the art, however, that the clamp pads 114 may be coupled to the body 103 in any of a variety of ways so long as the clamp pads 114 are movable relative to the body 103, as described, and are prevented from being inadvertently decoupled from the body 103.

According to an exemplary embodiment, the proximal portion 110 of the body 103 also includes a tape hook 138 extending along an exterior thereof. The tape hook 138 may be positioned along the exterior of the proximal portion 110, between the clamp pads 114 in, for example, circumferential alignment therewith. The tape hook 138 is configured so that an end of a length of tape may be hooked onto the tape hook 138 so that tension may be applied to and maintained on the tape as it is wound about the proximal portion 110, over the clamp pads 114, to move and maintain the clamp pads 114 in a radially inward configuration (i.e., the second position), in which the clamp pads 114 grip the endoscope 104 therebetween.

Additionally, the proximal portion 110 may also include a pair of channels 140 extending through the wall 116 of the proximal portion 110 from a proximal end 142 of the proximal portion 110 to a distal end 144 of the proximal portion 110. Each of the channels 140 is configured to engage the distal end 146 of a corresponding one of the tubular structures 122, as will be described in further detail below. As shown in FIG. 3, a proximal portion 150 of each of the channels 140 of this embodiment includes an optional a groove-like configuration along the exterior surface of the proximal portion 110 such that the channels 140 are laterally open to an exterior of the body 103. In an exemplary embodiment, a proximal portion of each of the channels 140 extends along a curved or is angled relative to the longitudinal axis of the body 103. In an exemplary embodiment, distal portions 158 of the channels 140 remain enclosed and extend substantially parallel to the longitudinal axis of the body 103.

As described above, the distal portion 112 of the body 103 is configured so that the clip 108 is mountable thereover, stretched into an open configuration, in which jaws 152 thereof are separated from one another so that tissue drawn into the channel 118 is drawn between the open jaws 152. The distal portion 112 of one embodiment has a diameter smaller than that of the proximal portion 110. In an exemplary embodiment, the distal portion 112 tapers from a maximum diameter at a proximal end 148 thereof to a minimum diameter at the distal end 128 of the body 103 so that a natural bias of the clip 108 may assist in moving the clip 108 distally off of the distal portion 112 so that the clip 108 closes (moves to the closed configuration) over target tissue that had previously been drawn into the channel 118. At least a portion of the distal portion 112 may be formed of a transparent material to facilitate visual observation of the clip 108 using, for example, an optical system of the endoscope 104 where the field of view of the optical system (e.g., including a lens on a distal face of a distal end of the endoscope 104) extends distally through the channel 118 and radially outward through the endcap 102 mounted over the distal end of the endoscope.

The distal portion 112 further includes a pair of grooves 154 extending along an exterior surface 156 of the distal portion 112, in longitudinal alignment with the distal portion 158 of the channels 140, so that control members 124 inserted through the tubular structures 122 extend therealong to the clip 108. The control members 124 may slide longitudinally within these grooves 154 as the control members 124 are moved longitudinally relative to the endcap 102 and the endoscope 104 to move the clip 108 relative thereto.

In an exemplary embodiment, the endcap 102 further comprises a seal 174 housed within the channel 118 of the body 103. The seal 174 is positioned within the channel 118, distally of the clamp pads 114 to engage a distal face of the endoscope 104 that is received within the channel 118 so that that the endoscope 104 is sealed thereagainst. Thus, vacuum force applied through a working channel of the endoscope 104 (i.e., where the working channel opens into the channel 118 through an opening in the distal face of the endoscope) and through the channel 118 of the body 103 is sealed within the channel 118 to suction tissue into the channel 118. The seal 174 may have any of a variety of configurations so long as the seal 174 is configured to be sealed against the distal end 106 of the endoscope 104. In one example, the seal 174 is configured as a ring-shaped gasket. In another example, the seal 174 is configured a lip valve which, as will be understood by those of skill in the art, is configured to open when subject to a force in a selected direction.

In an exemplary embodiment, the endcap 102 further comprises a joint member 120 positioned proximally of the proximal end 126 of the body 103 and connected thereto via longitudinal structures. The joint member 120 is sized, shaped and configured to extend along a portion of a circumference of the endoscope 104 and, in one embodiment, includes an interior surface 160 extending along a corresponding curve. The joint member 120 also includes a pair of channels 162 extending therethrough from a proximal end 164 of the joint member 120 to a distal end 166 of the joint member 120. Each of the channels 162 is configured to receive a proximal end 168 of a corresponding one of the tubular structures 122 so that the proximal ends 168 of the tubular structures 122 are connected to one another via the joint member 120 to form a substantially Y-shaped joint.

The joint member 120 may also be used as another point of fixation at which the endcap 102 is fixed to the endoscope 104. In an exemplary embodiment, an exterior surface 170 of the joint member 120 also includes a tape hook 172 configured to engage an end of a length of tape. In use, the end of the tape may be hooked to the tape hook 172 and wound about the joint member 120 and a portion of the endoscope 104 along which the joint member 120 extends to directly affix the joint member 120 to the endoscope 104.

The tubular structures 122 extend between the joint member 120 to the body 103 of the endcap 102. As described above, the proximal end 168 of each of the tubular structures 122 is received and fixed within a corresponding one of the channels 162 extending through the joint member 120 while the distal end 146 of each of the tubular structures 122 is received and fixed within a corresponding one of the channels 140 extending through the proximal portion 110 of the body 103. Thus, as shown in FIG. 5, control members 124 for controlling a movement of the clip 108 may extend through the tubular structures 122 to be releasably coupled to the clip 108 mounted over the distal portion 112 of the body 103 of the endcap 102. In particular, each of the control members 124 in one embodiment extends longitudinally from a proximal end (not shown) accessible to a user of the system 100, along a length of the endoscope 104, through the tubular structure 122 to a distal end extending distally past a distal opening of the corresponding channel 140 of the body 103 to be releasably coupled to the clip 108. The proximal end of the control members 124 may, for example, be coupled to a handle member configured to actuate longitudinal movement of the control members 124 relative to the endcap 102.

As described above, the endcap 102 is configured to facilitate application of the clip 108 over target tissue. According to an exemplary embodiment, the endcap 102 facilitates application of the clip 108, which may be configured as an over-the-scope clip including a pair of jaws 152, where the jaws 152 are connected to one another via hinges 176. In an exemplary embodiment, each of the jaws 152 extends along a curve from a first end 178 to a second end 180 so that a first one of the hinges 176 connects the first ends 178 of each of the jaws 152 to one another, while a second one of the hinges 176 connects the second ends 10 of each of the jaws 152 to one another.

In an exemplary embodiment, the hinges 176 are spring biased, biasing the jaws 152 toward a closed configuration, in which the jaws 152 are moved toward one another. Each of the jaws 152 may include teeth or other gripping features to enhance, in the closed configuration, gripping of target tissue therebetween. When the clip 108 is mounted over the distal portion 112 of the endcap 102, however, the jaws 152 are stretched open apart from one another held open by opposing portions of the endcap 102 so that the exterior surface 156 of the distal portion 112 maintains the clip 108 in the open configuration. Thus, when the clip 108 is mounted over the body 103 of the endcap 102, target tissue may be drawn into the channel 118 of the body 103 via, for example, a suction force, so that the target tissue is received between the jaws 152. The clip 108 may then be moved distally off the of the body 103 so that the jaws 152 of the clip 108 are free to move, under the natural bias of the hinges 176, toward closed configuration to grip the target tissue therebetween.

Movement of the clip 108 from the open configuration to the closed configuration may be controlled via control members 124, distal ends 182 of which are releasably coupled to the jaws 152. In particular, each of the jaws 152 may include a connecting element 184 releasably connecting the jaw 152 to a corresponding one of the distal ends 182 of the control members 124. According to an exemplary embodiment, the control members 124 may also be configured to facilitate repositioning of the clip 108 if, upon a review of an initial placement of the clip 108, an operator (e.g., physician) of the system 100 determines that the clip 108 has not been clipped over the target tissue as desired. Repositioning of the clip 108 may also be achieved via the control members 124, which may be moved longitudinally relative to the endoscope 104 to facilitate a longitudinal movement of the clip 108 relative to the endcap 102.

The distal ends 182 of the control members 124 may be releasably connected to the jaws 152 via any of a number of mechanisms. In one example, the distal ends 182 may include enlarged portions, each of which is configured to pass through the connecting element 184 configured as a keyhole shaped opening extending through a corresponding one of the jaws 152 when it is desired to finally deploy the clip 108. In another example, graspers at the distal ends 182 grasp corresponding connecting elements 184 of the jaws 152, releasing the connecting elements 184 from the graspers when it is desired to finally deploy the clip 108. In yet another example, the distal ends 182 of the control members 124 include hooks which disengage the connecting elements 184 when it is desired to finally deploy the clip 108. It will be understood by those of skill in the art that the distal ends 182 of the control members 124 and the connecting elements 184 of the jaws 152 may have any of a variety of configurations, so long as the distal ends 182 are configured to release the connecting elements 184 when the operator determines that the clip 108 has been clipped over the target tissue, as desired.

According to an exemplary method utilizing the system 100, the endcap 102 may be mounted over the distal end 106 of the endoscope 104, as described above. In particular, the proximal portion 110 of the body 103 is mounted over the distal end 106 of the endoscope 104. In an exemplary embodiment, the distal end 106 is inserted into the channel 118 of the body 103 until the distal end 106 abuts the seal 174 so that a seal is provided between the channel 118 and the endoscope 104.

As shown in FIG. 7, tape 186 is then wound about the proximal portion 110 by hooking an end thereof on the tape hook 138 so that the tape 186 may be tensioned as it is wound about the proximal portion 110, over the clamp pads 114, to move the clamp pads 114 radially inward from the first position which permits insertion of the endoscope 104 therebetween, and the second position, in which the clamp pads 114 are moved radially inward to grip the endoscope 104. As the operator of the system 100 continues (e.g., physician) to wind the tape 186 over the proximal portion 110, the tape 186 maintains the clamp pads 114 in the second position so that the body 103 of the endcap 102 is affixed over the distal end 106 of the endcap 102.

As described above, in an exemplary embodiment, the joint member 120 of the endcap 102 may also provide another point of fixation of the endcap 102 to the endoscope 104. The joint member 120, however provides a direct fixation to the endoscope 104. Similarly to the body 103, tape 188 may be hooked on the tape hook 172 of the joint member 120, tensioned, and wound about both the joint member 120 and the endoscope 104 to fix the joint member 120 thereto.

Once the endcap 102 has been mounted over the distal end 106 of the endoscope 104, the clip 108, mounted over the distal end 106 of the endcap 102, is inserted into the body (e.g., through a body lumen such as, for example, the GI tract) to a target area adjacent to tissue to be treated. According to an exemplary embodiment, the clip 108 is mounted over the distal portion 112 in the open configuration, with jaws 152 thereof extending over opposing sides of the distal portion 112 so that the jaws 152 are separated from one another. The endcap 102 and the clip 108 are then guided to a position adjacent to the tissue to be clipped using, for example, a visualization system of the endoscope 104.

In an exemplary embodiment, when the endcap 102 is positioned as desired (e.g., abutting the target tissue), the target tissue is drawn into the channel 118 via vacuum force applied through the endoscope 104 and the channel 118 of the body 103 to draw the target tissue between the jaws 152 of the clip 108. Although the exemplary embodiment describes a vacuum force applied through the channel 118 to draw tissue between the jaws 152 of the clip 108, it will be understood by those of skill in the art that tissue may be drawn between the jaws 152 using any of a variety of known methods including, for example, tissue graspers that are passed to the target area via a working channel of the endoscope 104.

Once the target tissue has been drawn between the jaws 152, the clip 108 is moved toward the closed configuration by moving the control members 124 which, as described above, extend from the proximal end to the distal end 182 connected to the clip 108, proximally relative to the endcap 102 and the endoscope 104. As will be understood by those of skill in the art, this releases tension along the control members 124 so that the clip 108, under its natural bias, is slid distally off the distal portion 112 of the body 103 toward the closed configuration, gipping the tissue between the jaws 152. As will also be understood by those of skill in the art, the control members 124 may slide along the grooves 154 of the distal portion 112 of the body 103 as the clip 108 is being moved between the open and the closed configurations.

According to an exemplary embodiment, this initial placement of the clip 108 may be reviewed by the operator prior to a final deployment thereof. It will be understood by those of skill in the art that, in these embodiments, the clip 108 remains tethered to the control members 124, as shown in FIG. 8, even when in this initial closed configuration. As described above, at least a portion of the distal portion 112 of the endcap 102 may be transparent to enhance visibility of the clip 108 and the surrounding tissue (via the visualization system of the endoscope 104) through the channel 118 and through the transparent endcap 102. In some embodiments, the operator may draw the endoscope 104 proximally away from the closed clip 108, while the clip 108 remains tethered to the control members 124, to further enhance the visibility of the clip 108.

If the operator determines that the clip 108 is not clipped over the target tissue, as desired, the operator may move the clip 108 from the closed configuration back to the open configuration by drawing the clip 108 proximally over the distal portion 112 of the body 103 of the endcap 102, by drawing the control members 124 proximally until the jaws 152 of the clip 108 once again extend over the opposing sides of the distal portion 112 in the open configuration. Since the endcap 102 and the body 103, in particular, is fixed to the distal end 106 of the endoscope 104 via the clamp pads 114, as described above, the endcap 102 is able to withstand the high forces applied to the endcap 102 as the clip 108 is pulled back over the body 103 to re-open the clip 108. In addition, as the clip 108 is moved from the closed configuration toward the open configuration, tissue may be retained in the channel 118 of the body 103 via the vacuum force applied therethrough.

The clip 108 may be moved between the open and the closed configuration, as many times as necessary, until the operator determines that the target tissue has been clipped as desired. Once the operator makes this determination, the distal ends 182 may be released from the corresponding connecting elements 184 of the clip 108 to finally deploy the clip. As described above, the control members 124 may release the clip 108, finally deploying the clip 108, via any of a variety of known release mechanisms.

Although the exemplary endcap 102 of the system 100 is specifically described and shown as being utilized for over-the-scope clipping of tissue, it will be understood by those of skill in the art that the endcap 102 may be similarly adapted for other endoscopic applications in which a tissue acquisition is required. For example, the endcap 102 may also be configured to facilitate a full thickness resection of tissue. In this embodiment, a length of the distal portion 112 of the endcap 102 may be selected so that a portion of the channel 118 extending therethrough is sized, shaped and configured to permit the acquisition of a full thickness portion of tissue. It will be understood by those of skill in the art that a full thickness resection would require a greater volume of tissue to be received within the channel 118 relative to procedures involving a simple hemostasis. Thus, in embodiments, in which the endcap 102 is being used for full thickness resection, the distal portion 112 may have a longer length to increase a volume of the channel 118 within which tissue may be received. Upon the acquisition of tissue, other medical instruments (e.g., snare) may be passed through the working channel of the endoscope and into the channel 118 to facilitate the resection of the acquired tissue.

It will be apparent to those skilled in the art that various modifications may be made in the present disclosure, without departing from the scope of the disclosure. Furthermore, those skilled in the art will understand that the features of any of the various embodiments may be combined in any manner that is not inconsistent with the description and/or the functionality of the embodiments.

1-15. (canceled)

16. An endcap device for clipping tissue, comprising:
a body extending longitudinally from a proximal end to a distal end and including a channel extending longitudinally therethrough, a proximal portion of the body sized shaped and configured to receive a distal end of an endoscope therein, and distal portion of the body sized and shaped so that a clip is mountable thereover, in an open configuration; and
a pair of clamp pads movably received within openings extending laterally through a wall along the proximal portion of the body, the pair of clamp pads movable from a first position in which the endoscope is receivable within the channel of the body between the pair of clamp pads, and a second position, in which the clamp pads are moved radially inward so that a portion thereof extend into the channel to grip a portion of the endoscope receivable therebetween.

17. The device of claim 16, further comprising:
a joint member sized, shaped, and configured to be positioned along a portion of an exterior surface of the endoscope, proximally of the body; and
a pair of tubular structures connecting the joint member to the proximal portion of the body, the pair of tubular structures configured to receive control members for controlling a movement of the clip mounted over the distal portion of the body.

18. The device of claim 16, further comprising:
a seal extending in the channel of the body, distally of the pair of clamp pads, the seal configured to seal the channel of the body against a distal face of the endoscope so that a vacuum force may be applied therethrough.

19. The device of claim 18, wherein the seal includes one of a seal gasket and a lip valve.

20. The device of claim 16, wherein the proximal portion includes a tape hook along an exterior surface thereof, between the pair of clamp pads and in alignment therewith.

21. The device of claim 17, wherein the joint member includes a tape hook configured to engage an end of a tape to facilitate attachment of the joint member to the exterior surface of the endoscope.

22. The device of claim 16, wherein each of the pair of clamp pads extends along a curve corresponding to a curvature along a portion of the channel of the body.

23. The device of claim 16, wherein an interior surface of each of the pair of clamps includes gripping structures for engaging a portion of the endoscope received therebetween.

24. The device of claim 16, wherein the distal portion of the body tapers from a proximal end thereof to a distal end thereof and includes grooves extending longitudinally along an exterior surface thereof.

25. A clipping system for treating tissue, comprising:
an endoscope extending longitudinally from a proximal end to a distal end;
an endcap including a body extending longitudinally from a proximal end to a distal end, the body including a channel extending longitudinally therethrough, a proximal portion of the body sized shaped and configured to receive the distal end of the endoscope therein and a distal portion of the body an insertion device therein, a pair of clamp pads movably received within openings extending laterally through a wall of the proximal portion of the body, the pair of clamp pads movable from a first position in which the endoscope is receivable within the channel of the body between the pair of clamp pads, and a second position, in which the clamp pads are moved radially inward to grip a portion of the endoscope received therebetween; and
a clip mountable over a distal portion of the body of the endcap, in an open configuration, in which jaws of the clip extend over opposing sides of the distal portion of the endcap so that the jaws are separated from one another, the jaws of the clip movable between the open configuration, and a closed configuration, in which the jaws are moved toward one another, the pair of jaws connected to one another via hinges biasing the clip toward the closed configuration.

26. The system of claim 25, wherein the endcap further includes a joint member connected to the proximal portion of the body via a pair of tubular structures, the tubular structures extending from proximal ends, connected to one another via the joint member to form a substantially Y-shape, to distal ends connected to the proximal portion of the body.

27. The system of claim 26, further comprising a pair of control members extending from proximal ends accessible to an operator of the system, along a length of the endoscope, through the pair of tubular structures to a distal end releasably connected to jaws of the clip to move the clip between the open configuration, the closed configuration, and a final deployed configuration.

28. The system of claim 26, wherein the joint member includes a tape hook configured to engage an end of a tape to facilitate attachment of the joint member to an exterior surface of the endoscope.

29. The system of claim 25, wherein the endcap includes a seal extending in the channel of the body, distally of the pair of clamp pads, to seal the channel of the body against a distal face of the endoscope so that a vacuum force may be applied therethrough.

30. The system of claim 29, wherein the proximal portion of the body of the endcap includes a tape hook along an exterior surface thereof, between the pair of clamp pads, in alignment therewith.

31. A method for treating tissue, comprising:
mounting an endcap over a distal end of an endoscope so that the distal end of the endoscope is received within a channel of a body of the endcap via a proximal portion of the endcap;
moving clamp pads received within openings through a wall of the proximal portion of the body of the endcap from a first position, in which the distal end of the endoscope is receivable within the channel between the clamp pads, and a second position, in which the clamp pads extend into the channel to grip the endoscope; and
fixing the clamp pads toward the second position so that the endcap is fixed over the distal end of the endoscope.

32. The method of claim 31, wherein moving the clamp pads from the first position toward the second position and fixing the clamp pads toward the second position includes winding a length of tape about the proximal portion of the body, about the clamp pads, to move and maintain the clamp pads in a radially inward configuration.

33. The method of claim 32, wherein the tape is wound about the proximal portion by hooking an end of the tape to a tape hook along an exterior of the proximal portion so that the tape is tensioned as the tape is wound about the proximal portion of the body.

34. The method of claim 31, further comprising:
fixing a joint member of the endcap directly to the endoscope, proximally of the body, the joint member connected to the body of the endcap via tubular structures configured to receive control members for controlling a movement of a clip relative to the endcap.

35. The method of claim 34, further comprising:
inserting the endcap, with the clip mounted over a distal portion thereof in an open configuration, to a target area within a body lumen;
drawing target tissue between jaws of the clip via a suction force applied through the endoscope and through the channel of the body of the endcap;
moving the clip between the open configuration, and a closed configuration, in which the clip is moved distally off of the distal portion of the body of the endcap so that the jaws move toward one another under a natural bias, wherein movement of the clip between the open and closed configurations is controlled via control members extending from proximal ends, along a length of the endoscope, through the tubular structure of the endcap to distal ends releasably coupled to the clip; and
finally deploying the clip, when it is determined that the clip is clipped over the target tissue as desired, by releasing the clip from the control members.
